(19) United States
(12) Patent Application Publication
(10) Pub. No.: US 2024/0425790 A1
(43) Pub. Date: Dec. 26, 2024

(54) MULTI-MODE MAGNETIC TREATMENT DEVICE COMBINING PERMANENT MAGNETIC FIELD AND ELECTROMAGNETIC FIELD

(71) Applicant: TONGJI UNIVERSITY, Shanghai (CN)

(72) Inventors: Yu CHENG, Shanghai (CN); Yali LIU, Shanghai (CN)

(73) Assignee: TONGJI UNIVERSITY, Shanghai (CN)

(21) Appl. No.: 18/684,692

(22) PCT Filed: Mar. 15, 2023
(86) PCT No.: PCT/CN2023/081484

§ 371 Date: Feb. 19, 2024

(30) Foreign Application Priority Data
Apr. 29, 2022 (CN) 202210472214.3

(51) Int. Cl. C12M 1/06; C12M 1/34

(52) U.S. Cl. CPC C12M 27/06; C12M 41/12; C12M 41/42

(57) ABSTRACT

A multi-mode magnetic treatment device combining a permanent magnetic field and an electromagnetic field includes a base, a sample stand, an electromagnetic mechanism, a permanent magnetic mechanism, a permanent magnetic mechanism motion drive unit, a sample stand ascent-descent drive unit and a control system. The sample stand, the electromagnetic mechanism and the permanent magnetic mechanism are mounted on the base. The electromagnetic mechanism and the permanent magnetic mechanism are connected to the control system. The permanent magnetic mechanism is mounted on the base through the permanent magnetic mechanism motion drive unit. The sample stand is mounted on the base through the sample stand ascent-descent drive unit. A magnetic field of the electromagnetic mechanism and a magnetic field of the permanent magnetic mechanism respectively act on the sample stand to apply a rotational permanent magnetic force and an alternating electromagnetic force to a sample.

### TECHNICAL FIELD

The invention relates to the technical field of magnetic treatment, in particular to a multi-mode magnetic treatment device combining a permanent magnetic field and an electromagnetic field.

### DESCRIPTION OF RELATED ART

Magnetic treatment fulfills the purpose of treating diseases by controlling physiological activities of patients with mechanical stimuli applied to cells by a force or force moment generated by magnetic materials under the action of a magnetic field. At present, the magnetic force has been applied to treatment study of tumors, cerebral diseases, skeletal-related diseases and other diseases because of its advantages of remote control, high temporal-spatial resolution and specificity.

In magnetic treatment, different magnetic fields, as the source of magnetic force, can be used to enable magnetic materials to make motions in different forms such as translatory motion and twisting to act on cells in different ways. Two types of magnetic fields are used for magnetic treatment: permanent magnetic field and electromagnetic field. For example, Cheon et al. targeted a magnetic material into hair cells of the inner ears and applied a square-wave magnetic field to the material to stimulate the hair cells to control the activity of the hair cells (ACS Nano 2014, 8, 6590-6598); and the research group made a great deal of study on the use of a rotational permanent magnetic field for treating tumors (Adv. Sci. 2020, 7, 1902933; Theranostics 2017, 7(6), 1735-1748).

In existing magnetic study, either the electromagnetic field or the permanent magnetic field is used for treatment, the treatment mode is monotonous, and treatment using both the permanent magnetic field and the electromagnetic field is rarely reported. However, magnetic conditions required for treating different diseases are different, and it is more necessary to combine different magnetic forces to treat complex and intractable diseases. Therefore, in order to satisfy the treatment requirements of different diseases and improve the treatment effect and efficiency, a multi-mode magnetic treatment device is urgently needed.

### BRIEF SUMMARY OF THE INVENTION

The objective of the invention is to overcome the abovementioned defects in the prior art by providing a multi-mode magnetic treatment device combining a permanent magnetic field and an electromagnetic field, which can realize multi-mode magnetic treatment.

The objective of the invention can be fulfilled through the following technical solution:

A multi-mode magnetic treatment device combining a permanent magnetic field and an electromagnetic field comprises a base, a sample stand, an electromagnetic mechanism, a permanent magnetic mechanism, a permanent magnetic mechanism motion drive unit, a sample stand ascent-descent drive unit and a control system;
the sample stand, the electromagnetic mechanism and the permanent magnetic mechanism are mounted on the base; the electromagnetic mechanism and the permanent magnetic mechanism are connected to the control system; the permanent magnetic mechanism is mounted on the base through the permanent magnetic mechanism motion drive unit; the sample stand is mounted on the base through the sample stand ascent-descent drive unit; and a magnetic field of the electromagnetic mechanism and a magnetic field of the permanent magnetic mechanism respectively act on the sample stand to apply a rotational permanent magnetic force and an alternating electromagnetic force to a sample.

Preferably, the magnetic treatment device is provided with a refrigerant cooling assembly connected to the control system; and the refrigerant cooling assembly is mounted on the base and used for cooling during electromagnetic operation.

More preferably, the refrigerant cooling assembly is specifically a water cooling assembly or a liquid helium cooling assembly.

Preferably, the electromagnetic mechanism is specifically an electromagnetic coil which is arranged around the sample stand.

More preferably, the electromagnetic coil is a common coil or a superconducting coil.

More preferably, the permanent magnetic mechanism is formed by one or more permanent magnets.

Preferably, the permanent magnetic mechanism motion drive unit comprises a magnet fixing member, a permanent magnet rotation drive module and a permanent magnet ascent-descent drive module; the permanent magnet ascent-descent drive module is fixed on the base; an output shaft of the permanent magnet ascent-descent drive module is connected to the permanent magnet rotation drive module; the magnet fixing member is arranged on an output shaft of the permanent magnet rotation drive module; and the permanent magnetic mechanism is fixed on the magnet fixing member.

More preferably, the permanent magnet rotation drive module and the permanent magnet ascent-descent drive module are both motors.

Preferably, the control system is used for controlling motions and electromagnetic outputs of the permanent magnetic mechanism motion drive unit and the sample stand ascent-descent drive unit, specifically including permanent magnetic motion modes, electromagnetic frequency, current magnitude and current direction.

More preferably, the permanent magnetic motion modes include jumping, a rotational motion mode, a rolling motion mode and a gyrational motion mode.

Compared with the prior art, the invention has the following beneficial effects:

The invention can realize multi-mode magnetic treatment: in the multi-mode magnetic treatment device provided by the invention can synchronously or respectively apply rotational permanent magnetic force and alternating electromagnetic force to a magnetic material, combines the advantage of high intensity of the permanent magnetic field and the advantage of diversity of electromagnetic mode, and can apply multiple magnetic stimuli, including jumping, rotation, rolling and gyration, on a sample to realize multi-mode magnetic treatment.

### BRIEF DESCRIPTION OF THE SEVERAL VIEWS OF THE DRAWINGS

FIG. 1 is a schematic structural diagram of a multi-mode magnetic treatment device according to the invention;

FIG. 2 is a schematic diagram of a rotational motion mode of a magnetic material in one embodiment of the invention;

FIG. 3 is a schematic diagram of a jumping motion mode of the magnetic material in one embodiment of the invention;

FIG. 4 is a schematic diagram of a gyrational motion mode of the magnetic material in one embodiment of the invention.

### REFERENCE SIGNS

1, base; 2, refrigerant cooling assembly; 3, a sample stand ascent-descent drive unit; 4, sample stand; 5, electromagnetic coil; 6, permanent magnetic mechanism; 7, magnet fixing member; 8, permanent magnet rotation drive module; 9, permanent magnet ascent-descent drive unit; 10, control system; 11, magnetic material; 12, magnetization direction.

### DETAILED DESCRIPTION OF THE INVENTION

The technical solutions of the embodiments of the invention will be clearly and completely described below in conjunction with accompanying drawings of the embodiments. Obviously, the embodiments in the following description are merely illustrative ones, and are not all possible ones of the invention. All other embodiments obtained by those ordinarily skilled in the art according to the following ones without creative labor should also fall within the protection scope of the invention.

### Embodiment 1

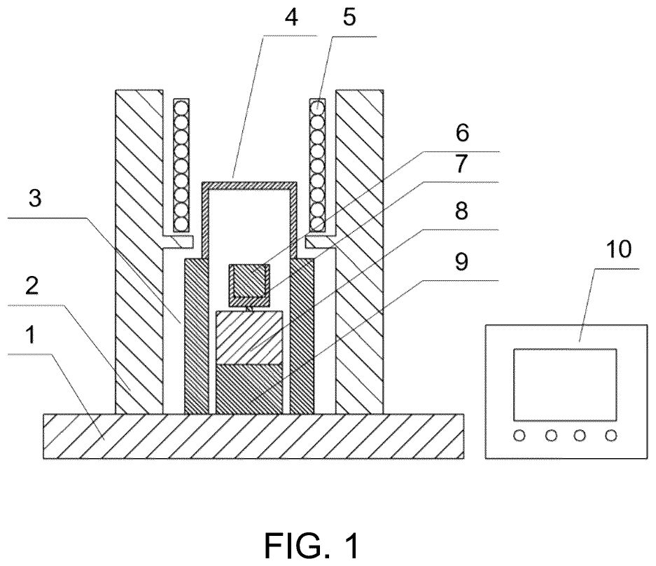

A multi-mode magnetic treatment device combining a permanent magnetic field and an electromagnetic field, the structure of which is shown in FIG. 1, comprises a base 1, a sample stand 4, an electromagnetic mechanism, a permanent magnetic mechanism 6, a permanent magnetic mechanism motion drive unit, a sample stand ascent-descent drive unit 3 and a control system 10. The sample stand 4, the electromagnetic mechanism and the permanent magnetic mechanism 6 are mounted on the base 1, the electromagnetic mechanism and the permanent magnetic mechanism 6 are connected to the control system 10, the permanent magnetic mechanism 6 is mounted on the base 1 through the permanent magnetic mechanism motion drive unit, the sample stand 4 is mounted on the base 1 through the sample stand ascent-descent drive unit 3, and a magnetic field of the electromagnetic mechanism and a magnetic field of the permanent magnetic mechanism 6 respectively act on the sample stand 4 to apply a rotational permanent magnetic force and an alternating electromagnetic force to a sample.

The magnetic treatment device is provided with a refrigerant cooling assembly 2 connected to the control system, and the refrigerant cooling assembly 2 is mounted on the base 1 and used for cooling during electromagnetic operation.

The refrigerant cooling assembly 2 is specifically a water cooling assembly or a liquid helium cooling assembly.

The electromagnetic mechanism is specifically an electromagnetic coil 5 which is arranged around the sample stand 4.

The electromagnetic coil 5 is a common coil or a superconducting coil.

The permanent magnetic mechanism 6 is formed by one or more permanent magnets.

The permanent magnetic mechanism motion drive unit comprises a magnet fixing member 7, a permanent magnet rotation drive module 8 and a permanent magnet ascent-descent drive module 9, wherein the permanent magnet ascent-descent drive module 9 is fixed on the base 1, an output shaft of the permanent magnet ascent-descent drive module 9 is connected to the permanent magnet rotation drive module 8, the magnet fixing member 7 is arranged on an output shaft of the permanent magnet rotation drive module 8, and the permanent magnetic mechanism 6 is fixed on the magnet fixing member 7.

The permanent magnet rotation drive module 8 and the permanent magnet ascent-descent drive module 9 are both motors.

The control system 10 is used for controlling motions and electromagnetic outputs of the permanent magnetic mechanism motion drive unit and the sample stand ascent-descent drive unit 3, specifically including permanent magnetic motion modes, electromagnetic frequency, current magnitude and current direction.

In this embodiment, the permanent magnetic motion modes include a jumping motion mode, a rotational motion mode, a rolling motion mode and a gyrational motion mode.

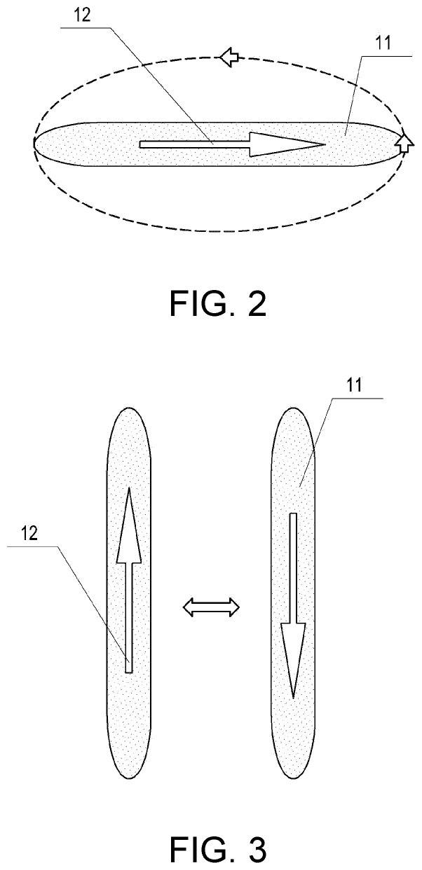

FIG. 2-FIG. 4 illustrate different motion modes of an electromagnetic material 11, and the magnetic material 11 and the magnetization direction 12 are as shown in FIG. 2-FIG. 4.

### Embodiment 2

In this embodiment, the magnetic material is made to rotate as shown in FIG. 2. In this mode, cells or tissues can be horizontally beaten. The specific operation is as follows:
(1) The multi-mode magnetic treatment device shown in FIG. 1 is used, and the sample stand is adjusted to a position close to the permanent magnet;
(2) The magnetic material is placed at the center of the sample stand above the permanent magnet;
(3) As the permanent magnet start to rotate horizontally, the magnetic line of force at the position where the magnetic material is located rotates in the horizontal plane, the magnetic material also rotates in the horizontal plane under the influence of the magnetic field, the motion mode of the magnetic material is the rotational motion mode shown in FIG. 2, cells or tissues can be horizontally beaten in this mode, and the beating frequency can be adjusted by adjusting the rotational frequency of the permanent magnet.

### Embodiment 3

In this embodiment, the magnet material is made to jump, as shown in FIG. 3. In this mode, cells or tissues can be beat like a drum. The specific operation is as follows:
(1) The multi-mode magnetic treatment device shown in FIG. 1 is used, the sample stand is adjusted to the center of the coil, and the permanent magnet is adjusted to the lowest position;
(2) The magnetic material is placed at the center of the sample stand;
(3) A current which is constant in magnitude and variable in direction is input to the coil, at this moment, the magnetic line of force at the position where the magnetic material is located changes constantly in the vertical direction, and in response to a change of the external magnetic field, the magnetic material jumps in the vertical direction; at this moment, the motion mode of the magnetic material is the rotational motion mode shown in FIG. 2, cells or tissues can be beaten like a drum in this mode, the beating frequency can be adjusted by adjusting the change frequency of the current, and the beating force can be adjusted by adjusting the amplitude of the current.

### Embodiment 4

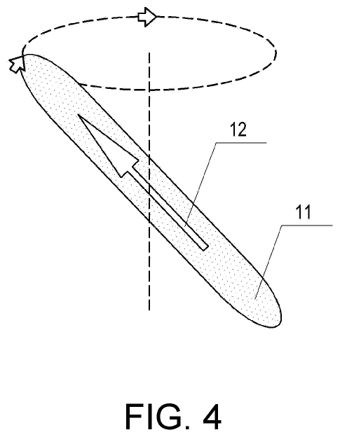

In this embodiment, the magnetic material is made to gyrate, as shown in FIG. 4. In this mode, a three-dimensional stirring force can be applied to cells or tissues. The specific operation is as follows:
(1) The multi-mode magnetic treatment device shown in FIG. 1 is used, and the sample stand and the permanent magnet are both adjusted to the center of the coil;
(2) The magnetic material is placed at the center of the sample;
(3) A permanent magnetic rotating button is turned on, a constant current is input to the coil, at this moment, an angle is formed between the magnetic line of force at the position where the magnetic material is located and the horizontal line, and the angle changes continuously; in response to a change of the external magnetic field, the magnetic material gyrates in three dimensions, as shown in FIG. 4; and in this mode, a three-dimensional stirring force can be applied to cells or tissues, the gyration angle and intensity of the magnetic material can be adjusted by adjusting the magnitude of the current, and the stirring frequency can be adjusted by adjusting the rotational frequency of the permanent magnet.

### Embodiment 5

In this embodiment, the magnetic material is made to rotate in different modes to apply a multi-mode force to cells or tissues. The specific operation is as follows:
(1) The multi-mode magnetic treatment device shown in FIG. 1 is used, and the sample stand and the permanent magnet are both adjusted to the center of the coil;
(2) The magnetic material is placed at the center of the sample stand;
(3) The rotation time of the permanent magnet and the duration of the current input to the coil are controlled to enable the magnetic material to switch between the horizontal rotation mode shown in FIG. 2 and the gyration mode shown in FIG. 4, such that a multi-mode magnetic force is applied to tissues or cells to improve the acting effect.

The above embodiments are merely specific ones of the invention, and the protection scope of the invention is not limited to the above embodiments. Various equivalent amendments or substitutions that can be easily thought out by any skilled in the art within the technical scope disclosed by the invention should fall within the protection scope of the invention. Therefore, the protection scope of the invention should be subject to the protection scope of the claims.

1. A multi-mode magnetic treatment device combining a permanent magnetic field and an electromagnetic field, comprising a base (1), a sample stand (4), an electromagnetic mechanism, a permanent magnetic mechanism (6), a permanent magnetic mechanism motion drive unit, a sample stand ascent-descent drive unit (3) and a control system (10),
wherein the sample stand (4), the electromagnetic mechanism and the permanent magnetic mechanism (6) are mounted on the base (1); the electromagnetic mechanism and the permanent magnetic mechanism (6) are connected to the control system (10); the permanent magnetic mechanism (6) is mounted on the base (1) through the permanent magnetic mechanism motion drive unit; the sample stand (4) is mounted on the base (1) through the sample stand ascent-descent drive unit (3); and a magnetic field of the electromagnetic mechanism and a magnetic field of the permanent magnetic mechanism (6) respectively act on the sample stand (4) to apply a rotational permanent magnetic force and an alternating electromagnetic force to a sample.

2. The multi-mode magnetic treatment device combining a permanent magnetic field and an electromagnetic field according to claim 1, wherein the magnetic treatment device is provided with a refrigerant cooling assembly (2) connected to the control system; and the refrigerant cooling assembly (2) is mounted on the base (1) and used for cooling during electromagnetic operation.

3. The multi-mode magnetic treatment device combining a permanent magnetic field and an electromagnetic field according to claim 2, wherein the refrigerant cooling assembly (2) is specifically a water cooling assembly or a liquid helium cooling assembly.

4. The multi-mode magnetic treatment device combining a permanent magnetic field and an electromagnetic field according to claim 1, wherein the electromagnetic mechanism is specifically an electromagnetic coil (5) which is arranged around the sample stand (4).

5. The multi-mode magnetic treatment device combining a permanent magnetic field and an electromagnetic field according to claim 4, wherein the electromagnetic coil (5) is a common coil or a superconducting coil.

6. The multi-mode magnetic treatment device combining a permanent magnetic field and an electromagnetic field according to claim 1, wherein the permanent magnetic mechanism (6) is formed by one or more permanent magnets.

7. The multi-mode magnetic treatment device combining a permanent magnetic field and an electromagnetic field according to claim 1, wherein the permanent magnetic mechanism motion drive unit comprises a magnet fixing member (7), a permanent magnet rotation drive module (8) and a permanent magnet ascent-descent drive module (9); the permanent magnet ascent-descent drive module (9) is fixed on the base (1); an output shaft of the permanent magnet ascent-descent drive module (9) is connected to the permanent magnet rotation drive module (8); the magnet fixing member (7) is arranged on an output shaft of the permanent magnet rotation drive module (8); and the permanent magnetic mechanism (6) is fixed on the magnet fixing member (7).

8. The multi-mode magnetic treatment device combining a permanent magnetic field and an electromagnetic field according to claim 7, wherein the permanent magnet rotation drive module (8) and the permanent magnet ascent-descent drive module (9) are both motors.

9. The multi-mode magnetic treatment device combining a permanent magnetic field and an electromagnetic field according to claim 1, wherein the control system (10) is used for controlling motions and electromagnetic outputs of the permanent magnetic mechanism motion drive unit and the sample stand ascent-descent drive unit (3), specifically including permanent magnetic motion modes, electromagnetic frequency, current magnitude and current direction.

10. The multi-mode magnetic treatment device combining a permanent magnetic field and an electromagnetic field according to claim 9, wherein the electromagnetic mechanism and the permanent magnetic mechanism (6) apply the rotational permanent magnetic force and the alternating electromagnetic force to the sample to enable a magnetic material to jump, rotate and roll to realize multi-mode magnetic treatment.
